(19) United States
(12) Patent Application Publication
(10) Pub. No.: US 2026/0158258 A1
(43) Pub. Date: Jun. 11, 2026

(54) DURABLE PERCUTANEOUS LEFT VENTRICULAR ASSIST DEVICE

(71) Applicant: Nicholas J. Shea, Basking Ridge, NJ (US)

(72) Inventor: Nicholas J. Shea, Basking Ridge, NJ (US)

(21) Appl. No.: 19/179,123

(22) Filed: Apr. 15, 2025

Related U.S. Application Data
(60) Provisional application No. 63/638,543, filed on Apr. 25, 2024.

(51) Int. Cl. A61M 60/17; A61M 60/13; A61M 60/861; A61M 60/865; A61M 60/88

(52) U.S. Cl. CPC A61M 60/17; A61M 60/13; A61M 60/861; A61M 60/865; A61M 60/88

(57) ABSTRACT

In accordance with at least one aspect of this disclosure, a system for delivery and placement of an intracardiac device within a patient comprises, a deployment wire, a sheath, and a snare. The deployment wire is configured for percutaneous delivery of an intracardiac device to a cardiac chamber through a peripheral artery. The deployment wire has a proximal end portion and a distal end portion, where the proximal end portion has a formed tip and the distal end portion is configured to operatively connect to the intracardiac device.

### CROSS-REFERENCE TO RELATED APPLICATIONS

This application claims priority to and the benefit of U.S. Provisional Ser. No. 63/638,543, filed Apr. 25, 2024, the contents of which are incorporated by reference herein in their entirety.

### TECHNICAL FIELD

The present disclosure relates to intracardiac devices and more particularly to a placement, delivery, and stabilization system for a cardiac pump.

### BACKGROUND

Conventional cardiac mechanical assist devices fall into two categories: durable and temporary. Current percutaneous temporary heart pumps are typically used for short term temporary support but are not suitable for patients to be discharged home. As used herein, “short term” means days to weeks. Other commonly used temporary support devices include counter pulsatility intra-aortic balloon pumps, extracorporeal membrane oxygenation, and the like. Patients with such devices require ICU level inpatient care by specially trained staff. While these devices remain implanted, patients typically await one of three outcomes: heart transplantation, recovery of heart function and removal of the device, or implantation of a so-called “durable” long-term device. As used herein “durable” means the device stays implanted for years to decades and patients are able to leave inpatient care to resume life at home.

Among the patients who are not likely to, or show no signs of, recovery, the decisions between transplantation and implantable left ventricular assist device (LVAD) are complex, but generally are determined by eligibility criteria for transplantation (e.g., UNOS/INTERMACS scoring). Transplantation has superior long-term outcomes to LVAD, and while patients with LVADs may remain on the transplant list, because of current prioritization schema and the enormous supply and demand imbalance for heart transplantation, so-called “bridge to transplant,” LVAD patients are unlikely to ever get an organ. Otherwise, the rest of these LVAD patients are referred to as receiving “destination therapy.”

Implantation of currently available LVADs requires open surgery. To implant such conventional devices, an incision is made in the chest, the sternum is divided, the heart is exposed, and the device is implanted using incisions and sutures. It requires repair of the bony trauma involved, drainage tubes, and has self-evident morbidity. Beyond the obvious morbidity of these operations, the patients requiring implantation of LVADs are typically very sick, with very weak hearts, and usually some degree of other organ dysfunction due to prolonged states of hypoperfusion (low blood flow) related to chronic pump failure. They are at very high risk for complications with an open surgery. Another potential complication of undergoing open surgery is that, if these LVAD patients do ever receive a heart for transplantation, the procedure known to surgeons as “LVAD explant/heart transplant,” is one of the longest, most complex, riskiest, and highest percentage complication operations in heart surgery. So called “virgin chest” heart transplantation, on the other hand, is much lower risk, shorter, and relatively easy from a technical standpoint.

The conventional techniques have been considered satisfactory for their intended purpose. However, there is an ever-present need for improved systems and methods for the availability of a percutaneously implantable durable ventricular assist device. This disclosure provides a solution for this need.

### SUMMARY

In accordance with at least one aspect of this disclosure, a system for delivery and placement of an intracardiac device within a patient comprises, a deployment wire, a sheath, and a snare. The deployment wire is configured for percutaneous delivery of an intracardiac device to a cardiac chamber through a peripheral artery. The deployment wire has a proximal end portion and a distal end portion, where the proximal end portion has a formed tip and the distal end portion is configured to operatively connect to the intracardiac device.

The sheath is configured for percutaneous insertion into a cardiac chamber wall through a chest wall. The sheath includes: a tubular body extending along an axis and having a proximal end portion, distal end portion, and a flexible portion therebetween configured for axial movement to accommodate cardiac movement during placement and delivery of the intracardiac device in the cardiac chamber. The sheath is configured to be inserted through the chest wall and into the cardiac chamber wall such that the flexible portion is within the pericardium and proximate the chamber wall. The distal end portion of the sheath includes one or more anchoring features configured to actuate between a deployed position where the one or more anchoring features extend radially outward from the distal end portion of the sheath, and a retracted position where the one or more anchoring features are within the distal end portion.

The snare is configured to be passed through the sheath into the cardiac chamber for snaring the formed tip of the deployment wire to pull the deployment wire and intracardiac device through the peripheral artery and into the cardiac chamber. In certain embodiments, the snare can be a loop or hook feature configured to grab the formed tip of the deployment wire.

In certain embodiments, the proximal end portion of the sheath can include a one-way valve configured to allow insertion of deployment instruments (e.g., dilators, obturators, the snare, among others) and prevent egress of fluids from the cardiac chamber. In certain embodiments, the flexible portion of the sheath can be or include a bellows.

In certain embodiments, with the one or more anchoring features of the sheath are in the retracted position, an outer circumference of the one or more anchoring features can be flush with the distal end portion of the sheath. In certain embodiments, with the one or more anchoring features in the retracted position, the one or more anchoring features can be retracted entirely within the distal end portion of the sheath.

In certain embodiments, the proximal end portion of the sheath can include an actuator operatively connected to the one or more anchoring features configured to actuate the one or more anchoring features to move between the retracted position and the deployed position. In certain embodiments, the actuator can be or include a push button actuator or the like. In certain embodiments, the one or more anchoring features can be configured to automatically deploy or retract, e.g., via spring bias.

In certain embodiments, the cardiac chamber is a first cardiac chamber, and the system further includes a deployable stent configured to be operatively connected to a proximal end of the intracardiac device for securing the intracardiac device within a cardiac valve or a second cardiac chamber during placement and operation of the intracardiac device within the cardiac chamber. The deployable stent is configured to move between a compressed position and a deployed position, where the deployable stent is configured to be in the compressed position during delivery of the intracardiac device, and where the deployable stent is configured to be deployed to the deployed position during placement and operation of the intracardiac device. In certain embodiments, the deployable stent is configured to automatically transition from the compressed to the deployed position, e.g., via spring bias.

In certain embodiments, the deployable stent comprises an annular portion configured to attach to and seat within a cardiac valve or the second cardiac chamber, the annular portion extending along an axis between a first end and a second end. One or more compression members can be operatively connected to the annular portion and extending radially inward from the outer diameter toward the axis and positioned axially between the first end and the second end of the annular portion. A central bore can be defined through or by the one or more compression members configured to accept the proximal end of the intracardiac device to hold the intracardiac device radially within a center of the cardiac valve or the second cardiac chamber during placement and delivery of the intracardiac device within the first cardiac chamber.

In certain embodiments, the one or more compression members can form a flexible membrane configured to allow axial and radial movement of the distal end of the intracardiac device with contraction and relaxation of the cardiac chamber without dislodgement of the intracardiac device or movement of the intracardiac device towards a cardiac chamber or valve wall. The flexible membrane is configured to allow blood flow to pass through the stent, around the pump. In certain embodiments, the annular portion and compression members are of a material having a spring force configured for self-deployment of the stent when placed within the cardiac valve or chamber.

In certain embodiments, the system further includes an attachment member configured to be operatively connected to the distal end of the intracardiac device to secure the distal end of the intracardiac device in the cardiac chamber and proximate the chamber wall. The attachment member can be further configured to provide a standoff distance between the distal end of the intracardiac device and the chamber wall, for example to prevent contact of the distal end of the intracardiac device with the chamber wall. This ensures a steady flow of blood enters the distal end of the pump.

In certain embodiments, the system can further include an occluder configured to be operatively connected to the distal end of the intracardiac device to occlude an aperture in the cardiac chamber wall made by the sheath during placement and operation of the intracardiac device. The occlude is configured to reducing bleeding from the chamber wall during placement of the intracardiac device system, and to aid in healing.

In certain embodiments, the system includes the intracardiac device. The intracardiac device can be or include a pump having a pump inlet at the distal end and a pump outlet at the proximal end. The deployable stent can be operatively connected to the proximal end of the pump for securing the pump within a cardiac valve or a second cardiac chamber during placement and operation of the pump within the cardiac chamber. The attachment member can be operatively associated with the distal end of the pump to secure the distal end of the pump in the cardiac chamber and proximate the chamber wall at a standoff distance from the chamber wall. In certain embodiments, the occlude, configured to occlude an aperture in the cardiac chamber wall made by the sheath during placement and operation of the intracardiac device, is included near the distal end of the pump.

In certain embodiments, each of the pump and the occluder are operatively associated with the deployment wire such that pulling the deployment wire via the snare through the aperture in the chest wall pulls the pump and occluder through the peripheral artery and into the cardiac chamber for placement in the cardiac chamber.

In certain embodiments, the intracardiac device is a left ventricular assist device, the cardiac chamber is a left ventricle, and the cardiac valve is an aortic valve. The sheath is configured to be introduced through the chest wall, through a left ventricle wall, and into the left ventricle transapically. In certain embodiments, the peripheral artery can be the femoral artery. In such embodiments, the deployment wire can be configured to be introduced though and into the femoral artery and passed through to the left ventricle via the aorta. The snare can be configured to be introduced through the sheath transapically to snare the deployment wire, whereby pulling the snare in turn externalizes the deployment wire through the sheath and pulls the pump through the femoral artery and through the ascending aorta such that: the occluder is inserted into the aperture, the pump inlet is positioned in the left ventricle proximate the left ventricle wall, the pump outlet is positioned in or proximate to the ascending aorta, and the deployable stent is positioned in the ascending aorta.

The pump further can further a drive wire configured to provide power to the pump, configured to be externalized through the aperture with the deployment wire. With the pump in place, an entirety of the pump, the sent, the attachment member, and the occluder are within the heart, and only the drive wire of the pump is externalized through the ventricular wall and through the chest wall.

In accordance with at least one aspect of this disclosure, each of the deployment wire, the snare, the intracardiac device, the stent, the sheath, the occluder can be included in a package forming a kit. The kit may also include one or more additional surgical instruments, such as additional sheaths, introducers, obturators, dilators, or the like. In certain embodiments, within the kit, the occluder and the intracardiac device can be operatively associated with the deployment wire, for example, the intracardiac device and occluder can be pre-loaded onto the deployment wire. In certain embodiments, the stent and attachment member can be pre-assembled onto the intracardiac device within the kit.

In accordance with at least one aspect of this disclosure, a system for delivery and placement of an intracardiac device can include a first sheath configured to be placed proximate a peripheral artery, a deployment wire configured for delivering the intracardiac device to a heart through the peripheral artery, wherein the deployment wire includes a formed tip, a second sheath configured for percutaneous transapical insertion into a cardiac chamber wall, and a snare configured to be passed through the transapical sheath for snaring the formed tip of the deployment wire to pull the deployment wire and intracardiac device attached thereto through the peripheral artery and into the heart.

In accordance with at least one aspect of this disclosure, an intracardiac device system can include, an intracardiac device having a first end and a second end opposite the first end configured to be proximate a chamber wall of the heart when deployed in the heart, a stent operatively connected to the intracardiac device at the first end configured to secure the intracardiac device within the heart at the first end, an attachment member operatively connected to the intracardiac device at the second end configured to secure the second end of the intracardiac device in a cardiac chamber at the chamber wall, an occluder operatively connected to the intracardiac device at the second end configured to promote attachment of the attachment member to the chamber wall while reducing bleeding from the chamber wall during placement of the intracardiac device system in the heart, a deployment sheath configured to be inserted into the chamber wall in a transapical approach via percutaneous incision, the deployment sheath configured to be placed during delivery of the intracardiac device system, and removed once delivery of the intracardiac device is complete, and a drive line operatively connected to the second end of the intracardiac device configured to externalize from the intracardiac device, through the chamber wall and the percutaneous incision, and attach to a computerized device and/or power source to provide power to and/or control the intracardiac device.

In accordance with at least one aspect of this disclosure, an intracardiac device can include a pump having an inlet end and an outlet end, a stent operatively connected at the outlet end end of the pump configured to secure the pump within the heart at the outlet end; an attachment member operatively connected to the inlet end configured to secure the second end of the pump in a chamber wall of a cardiac chamber at a standoff distance; and an occluder operatively associated with the inlet end of the pump configured to promote attachment of the attachment member to the chamber wall while reducing bleeding from the chamber wall during placement of the intracardiac device system in the heart.

In accordance with at least one aspect of this disclosure, a sheath configured for transapical insertion for placement of and intracardiac device can include, a tubular body having a proximal end portion, and distal end portion, and a flexible portion there between. In certain embodiments, the proximal end portion includes a one-way valve for allowing insertion of deployment instruments but prevents egress of fluids from the heart, the flexible portion is configured for axial movement to accommodate heart movement during placement and delivery of the intracardiac device, and the distal end portion includes one or more anchoring features controllable at the proximal end portion, the anchoring features configured to deploy during placement of the intracardiac device and retract once placement is complete.

In accordance with at least one aspect of this disclosure, a stent can include an annular portion configured to attach to and seat within a first cardiac valve or chamber, the annular portion extending along an axis between a first end and a second end, and a flexible membrane portion operatively connected to an inner diameter of the annular portion and extending radially inward from the outer diameter toward the axis, positioned axially between the first end and the second end of the annular portion. A central bore can be defined through the flexible membrane portion configured to accept an outlet end of an intracardiac device extending from a second cardiac chamber through a second cardiac valve, and into the first cardiac chamber or valve to hold the intracardiac device radially within a center of the first cardiac chamber or valve when the intracardiac device is placed in the heart. The flexible membrane portion can be configured to allow some axial and some radial movement of the intracardiac device to accommodate natural movement of the heart in use without dislodgement of the intracardiac device or movement of the intracardiac device towards a cardiac chamber or valve wall. In certain embodiments, the annular portion and flexible membrane portion can be of a material having a spring force configured for self-deployment of the stent when placed within the heart.

In accordance with at least one aspect of this disclosure, a method for delivery and placement of an intracardiac device system in a heart can include the steps of:
a) inserting a first sheath percutaneously proximate a peripheral artery;
b) inserting a deployment wire through the first sheath and into the peripheral artery and feeding the deployment wire through the peripheral artery into a cardiac chamber of the heart;
c) inserting a second sheath percutaneously and transapically into a wall of the cardiac chamber;
d) inserting a snare through the second sheath and into the cardiac chamber and snaring the deployment wire with the snare to externalize the deployment wire through the second sheath;
e) feeding the intracardiac device into the peripheral artery through the first sheath, and into the heart via the deployment wire being externalized through the second sheath, wherein pulling the intracardiac device into proper placement urges deployment of a stent in the heart to secure the intracardiac device into place;
f) deploying an occluder at the percutaneous incision at the chamber wall and deploying an attachment member to secure the intracardiac device to the chamber wall and stabilize the intracardiac device within the cardiac chamber;
g) externalizing a drive wire of the intracardiac device through the second sheath and connecting the drive wire to an external device for controlling and/or powering the intracardiac device;
h) removing the deployment wire through the first sheath, removing the first sheath, removing the second sheath; and
i) starting operation of the intracardiac device.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 is a partial cross-sectional view of a heart showing an embodiment of an intracardiac device system in accordance with this disclosure, where the intracardiac device placed in a heart of a patient;

FIG. 2 is a perspective view of an embodiment of an intracardiac device system in accordance with this disclosure, showing a stent, a pump, and an attachment member;

FIG. 3A is a perspective view of a sheath in accordance with this disclosure;

FIG. 3B is a perspective view of the sheath of FIG. 3A, showing a surgical instrument inserted into the sheath;

FIGS. 4-7 show sequential stages of a method of delivery and placement of the intracardiac device of FIG. 1 within the heart of the patient, wherein:

FIG. 4 shows insertion of the sheath into a cardiac chamber wall, and a deployment wire placed within a cardiac chamber proximate the sheath;

FIG. 5 shows a snare inserted through the sheath to snare the deployment wire;

FIG. 6 shows the snare being pulled through the sheath, in turn pulling the deployment wire and the intracardiac device operatively connected thereto, through the body and into the heart; and

FIG. 7 shows the intracardiac device in place, with the deployment wire removed, the sheath removed, and a drive wire of the intracardiac device externalized through the chest wall; and

FIG. 8 shows an embodiment of a kit in accordance with this disclosure.

### DETAILED DESCRIPTION

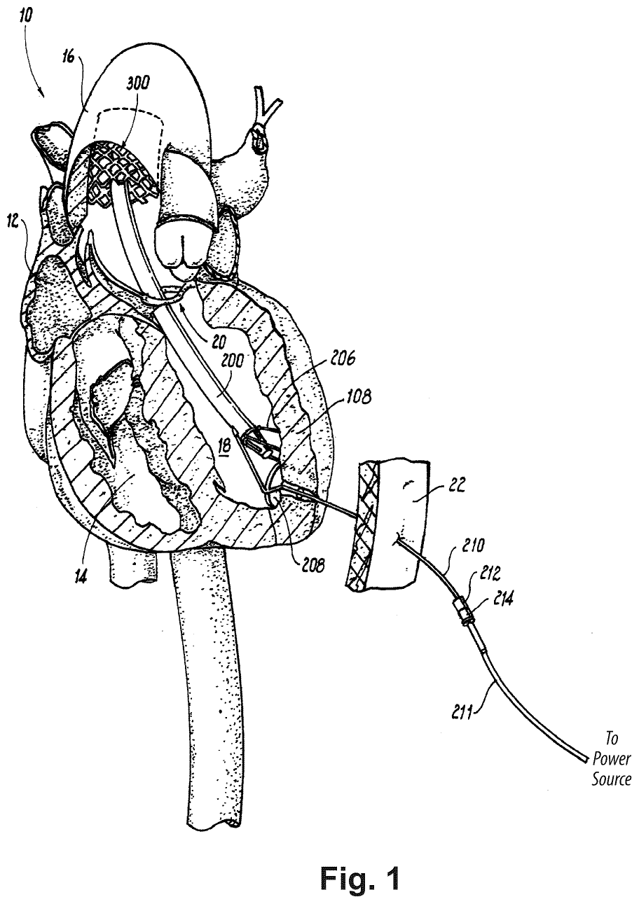

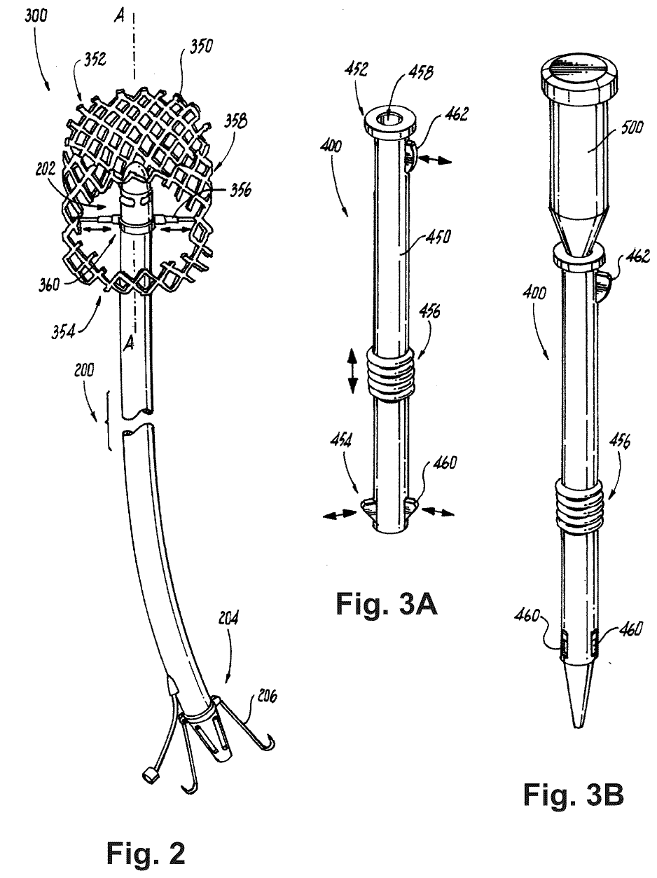

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1 and is designated generally by reference character 100. Other embodiments and/or aspects of this disclosure are shown in FIGS. 2-8.

The terms mechanical assist device, artificial heart pump, left ventricular assist device, biventricular assist device, and artificial heart do not always refer to the same thing but are often used interchangeably and refer to a category of mechanical pump devices used to treat patients with severe heart failure. Though there are differences terminologically in what these devices do and how they work, for the purposes of ease of explanation of this disclosure, the above reference terms as used herein can be interpreted to mean the same thing.

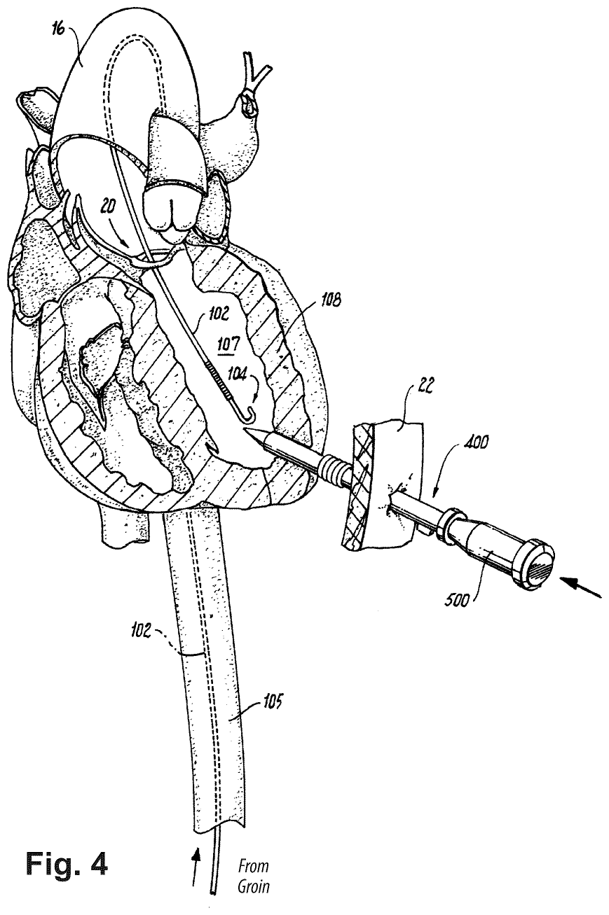

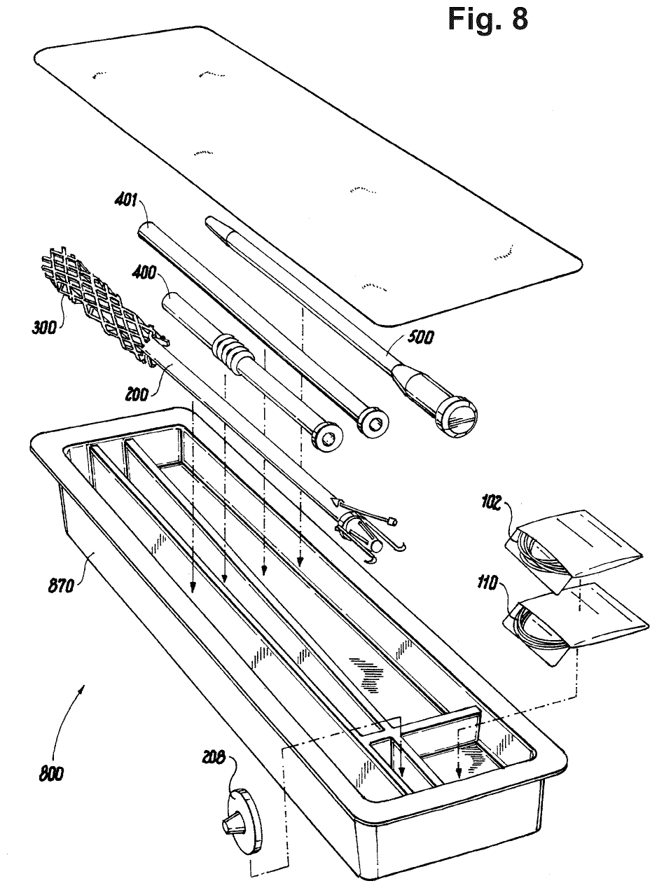

Referring to FIG. 1, in accordance with at least one aspect of this disclosure, a system 100 for delivery and placement of an intracardiac device 200 can include, a deployment wire 102 configured for delivering the intracardiac device 200 to a heart through the peripheral artery, the deployment wire having a formed tip 104, a sheath 400 configured for percutaneous transapical insertion into a cardiac chamber wall 108, and a snare 110 configured to be passed through the transapical sheath 106 for snaring the formed tip of the deployment wire to pull the deployment wire and intracardiac device 200 attached thereto through the peripheral artery and into the heart. FIG. 1 shows the intracardiac device 200 placed in the heart, and FIGS. 4-7 show the placement of the device 200, moving from a peripheral artery (not shown, labelled “from groin”), and into the heart via the deployment wire 102. FIG. 8 shows an embodiment of a kit 800, including the components of the system 100.

As shown in FIGS. 1 and 2, in certain embodiments, the intracardiac device 200 can include a stent 300 operatively connected at a proximal end 202 configured to secure the intracardiac device within the heart at the proximal end 202, an attachment member 206 operatively connected to a distal end 204 configured to secure the second end of the intracardiac device in a chamber wall 108 of a cardiac chamber. The intracardiac device 200 can also include an inbuilt plug/stopper or occluder 208. The occluder 208 can be operatively connected to the intracardiac device 200 at the distal end 204 configured to promote attachment of the attachment member to the chamber wall 108 while reducing bleeding from the chamber wall during placement of the intracardiac device system 100 in the heart. In certain embodiments, the occluder 208 can be percutaneously deployed.

With specific reference to FIG. 2, in certain embodiments, stent 300 is an inbuilt, uncovered, self-expanding stent frame at the proximal end 202 (e.g., an outflow) of the pump in the ascending aorta. The stent 300 and its attachment to the cardiac pump system 100 is distinguished from the prior art and conventional LVADs because, among other things, the stent 300 is percutaneously deployed in the ascending aorta and thus secures the pump in the aorta without requiring open chest surgery.

As shown in FIG. 2, the stent 300 can include an annular portion 350 configured to attach to and seat within a first cardiac valve or chamber, the annular portion extending along an axis A between a first end 352 and a second end 354. A flexible membrane portion, or compression members, 356 can be operatively connected to an inner diameter 358 of the annular portion and extend radially inward from the inner diameter 358 toward the axis A, positioned axially between the first end 352 and the second end 354 of the annular portion 350. While two compression members 356 are shown, in certain embodiments, a plurality of compression members forming a mesh membrane can be included. A central bore 360 can be defined through the flexible membrane 366 configured to accept an end 202 of the intracardiac device 200 extending from a second cardiac chamber through a second cardiac valve and into the first cardiac chamber or valve. The flexible membrane 366 can be configured to hold the intracardiac device radially within a center portion of the first cardiac chamber or valve when the intracardiac device is placed in the heart.

In certain embodiments, flexible membrane 366 or member 356 is configured to allow some axial movement and some radial movement of the intracardiac device 200 to accommodate natural beating movement of the heart without dislodgement of the intracardiac device 200 or movement of the intracardiac device towards a cardiac chamber or valve wall. In certain embodiments, the annular portion 350 and flexible member 356 can be of a material having a spring force configured for self-deployment of the stent when placed within the heart, e.g., when the stent 300 reaches a part of the heart having sufficient diameter for the stent to self-expand with the biased spring force of the respective materials such as a shape memory alloy.

With specific reference to FIGS. 3A and 3B, in certain embodiments, the sheath 400, can be a sheath configured for transapical insertion for placement of an intracardiac device, e.g., the intracardiac device 200. The sheath 400 can include a tubular body 450 having a proximal end portion 452, a distal end portion 454, and a flexible portion 456 therebetween. The proximal end portion 452 can include a one-way valve 458 for allowing insertion or introduction of deployment instruments (e.g., dilator/obturator 500, the snare 110, or other instruments), but which inhibits or prevents egress of fluids from the heart. The flexible portion 456 can include a bellows region 456 configured for flexibility in axial movement to accommodate heart movement during placement and delivery of the intracardiac device 200, e.g., normal beating motion.

The distal end portion 454 of the sheath 400 can include one or more anchoring features 460 controllable via actuator 462 at the proximal end portion 452. The anchoring features 460 can be configured to deploy during placement of the intracardiac device 200 and retract once placement is complete. The anchoring features 460 can be actuated via button 462 at the proximal end portion 452, for example, once the sheath 400 has been inserted into the chamber wall so that the sheath 400 is anchored during the placement of the intracardiac device 200, e.g., to maintain connection to the ventricular chamber despite normal physiologic motion.

In certain embodiments, the intracardiac device 200 can be or include a cardiac pump configured for placement within a left ventricle and extending into the ascending aorta. In such embodiments, the stent 300 of FIG. 2 can be configured to self-deploy in the ascending aorta to anchor the first end 202 of the pump in the ascending aorta, the proximal end 202 being a pump outlet end. The distal end 204 of the pump 200 is a pump inlet and can be configured for placement within the left ventricle 18. The transapical sheath 400 of FIG. 3A is inserted into the ventricular wall 108 during placement, and the occluder 208 of FIG. 1 and attachment member 206 are anchored into the ventricular wall 108. The attachment members 206 can be sized to provide a standoff distance between the pump inlet end 204 and the chamber wall 108. This prevents the pump from getting sucked down onto the ventricular wall 108, ensuring continuous and proper flow of blood from the ventricle through the pump and to the rest of the body.

Once the pump 200 is placed, the drive line 210 will externalize through the ventricular wall, and chest, without wrapping back through the heart. The drive line will provide a connection for power and control of the device 200 inside the heart from outside the chest. As shown in FIG. 1, one placed and operational, an entirety of the system 100 is within the heart, with the exception of the drive wire 210. The drive wire 210 is externalized through the chest wall proximate the ventricular wall to avoid extensive caballing positioned within the body, reducing chances for infection. This is different than conventional systems, which require components to wrap around the exterior of the heart, and the drive wire to be externalized in the abdomen. Embodiments of the system 100 therefore include a more compact and less complex system, reducing the chances of complications for the patient during placement and operation of the system.

In accordance with at least one aspect of this disclosure, a method for delivery and placement of an intracardiac device system (e.g., system 100) in a heart is described, and shown in FIGS. 4-7. While the method is shown and described with respect to inserting the device 200 though a peripheral artery to the left ventricle, one having ordinary skill in the art in view of this disclosure would appreciate that the method can be used for placement of the intracardiac device in any suitable location as needed or desired.

The method can include, inserting a first sheath percutaneously proximate a peripheral artery (not shown) and inserting a deployment wire 102 through the first sheath and into the peripheral artery, e.g., using standard fluoroscopy, echocardiographic, and ultrasound guidance. The deployment wire is fed through the peripheral artery, through the aorta 105, and into a cardiac chamber 107 of the heart as shown in FIG. 4 via the aorta.

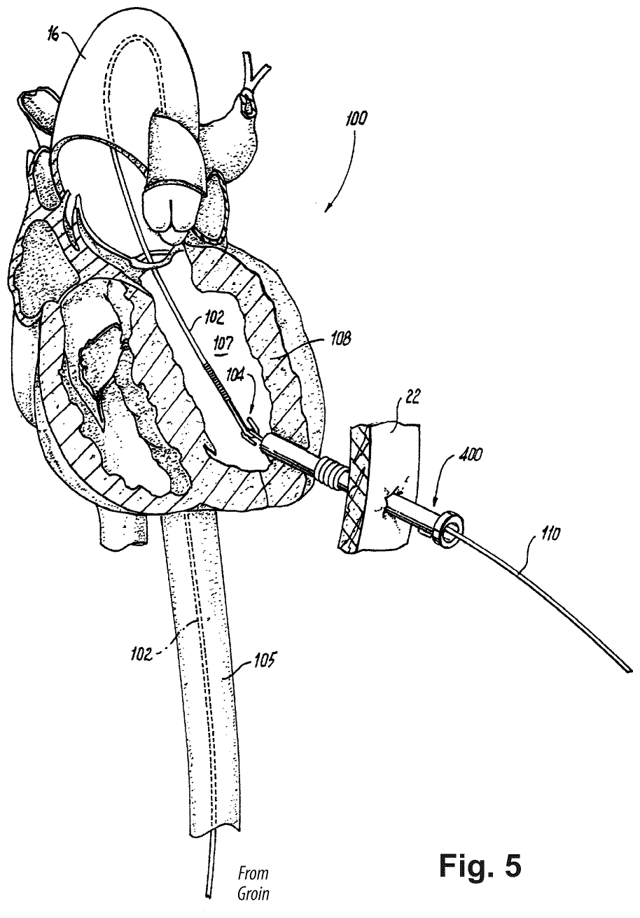

The method can further include, inserting a second sheath 400 percutaneously and transapically into a wall 108 of the cardiac chamber 107. Specifically, at the chest wall, using ultrasound, fluoroscopy, and echocardiography, a needle can be passed through the chest wall and into the cardiac chamber 107, typically at or near the apex. The second sheath 400 is passed over the needle into the chamber 107. The method can include inserting a snare 110 through the second sheath 400 and into the cardiac chamber 108 and snaring the deployment wire 102 with the snare 110 to externalize the deployment wire 102 through the second sheath, for example as shown in FIGS. 4-7. In FIG. 4, the deployment wire 102 has been introduced through the peripheral artery and fed through the body into the heart. The sheath 400 has been introduced through the chest wall 22 and into the cardiac chamber wall 18, allowing for introduction of the snare 100. In FIG. 4, a dilator or introducer 500 is shown in the sheath 400. In FIG. 5, the snare 110 is shown snaring the tip 104 of the deployment wire 102 in the chamber 107. This will allow a surgeon to pull the snare 110, and thus the deployment wire 102 out through the second sheath 400 to externalize the deployment wire 102.

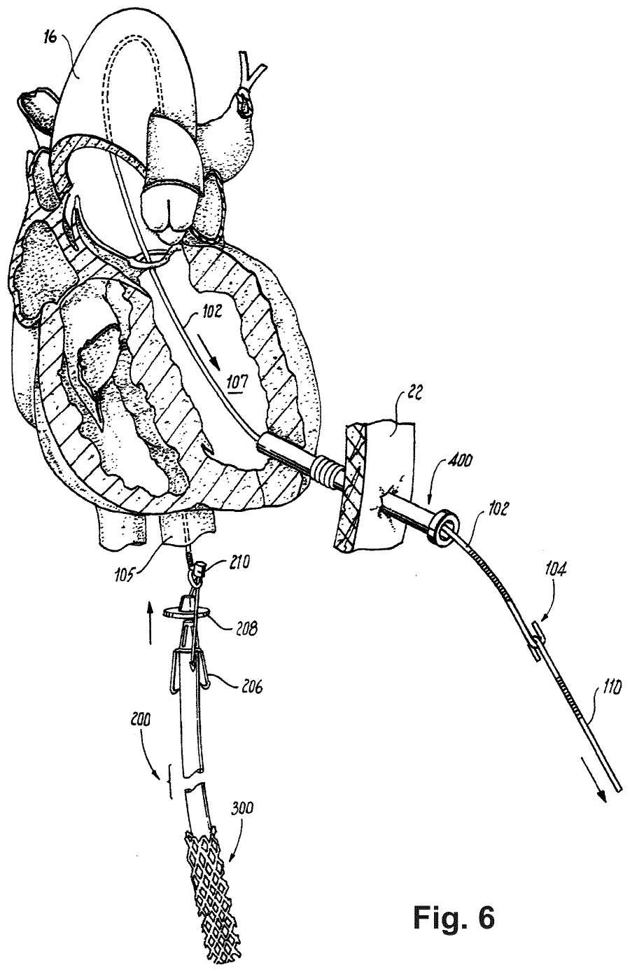

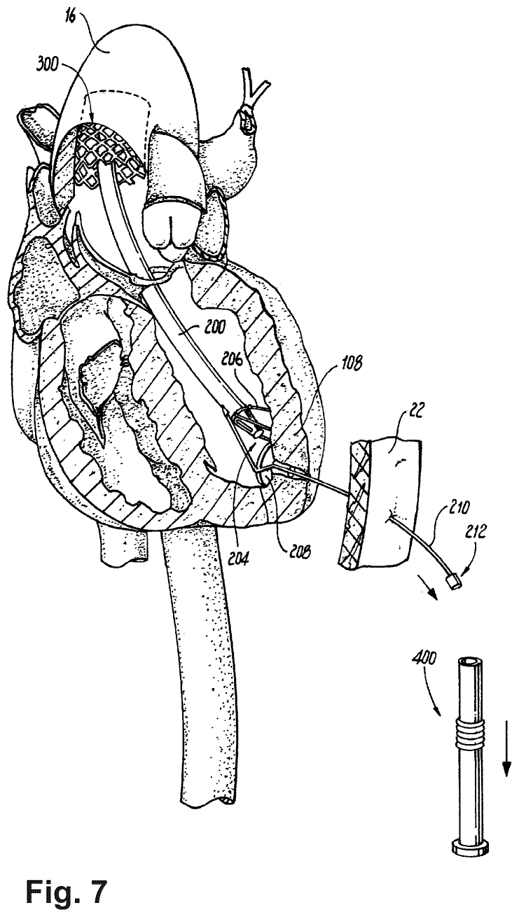

As shown in FIG. 6, the method can further include feeding the intracardiac device 200 into the peripheral artery through the first sheath, and into the heart via the deployment wire 102 being externalized through the second sheath 400. FIG. 6 shows the procedure after the snare 110 has pulled the deployment wire 102 through the second sheath 400 to bring the intracardiac device 200 into view. As the surgeon continues to pull the deployment wire 102, which is now externalized, the intracardiac device 200 will snake through the aorta 105 until its final placement with the pump outlet 202 placed in the ascending aorta, the pump inlet 204 placed in the chamber 107, and the device drive wire 210 is externalized through the sheath 400, which is shown in FIG. 7

Also shown in FIG. 7, the method can further include, deploying the occluder 208 at the percutaneous incision (e.g., an aperture) at the chamber wall 108 and deploying the attachment member 206 to secure the intracardiac device 200 to the chamber wall 108 and stabilize the intracardiac device 200 within the cardiac chamber 107. Pulling the intracardiac device 200 into proper placement urges deployment of the stent 300 in the ascending aorta to secure the pump into place once the device 200 is in place, the deployment wire 102 is removed through the peripheral artery, and a drive line 210 is connected to the computerized device/power source.

Once the intracardiac device 200 is secure, the method can include, externalizing the drive line 210 of the intracardiac device through the second sheath 400 and connecting the drive wire to an external device for controlling and/or powering the intracardiac device. The method can further include, removing the deployment wire 102 through the first sheath, removing the first sheath, removing the second sheath 400, and starting operation of the intracardiac device 200. FIG. 7 shows the final placement of the intracardiac device 200, the deployment wire being removed, and the drive line connected.

FIG. 8 shows an embodiment of a kit 800. In certain embodiments, all components for percutaneous delivery of the system 100 can be included in a single package 870. As shown in FIG. 8, the deployment wire 102 and occluder 208 are shown detached from the pump 200. However, in certain embodiments, each of the stent 300 and occluder 208 can be included in the package 870 already attached to the pump 200. In certain embodiments, the occluder 208 can be integral or inbuilt with the pump 200. Further, in certain embodiments, the pump 200 and its associated components, e.g., the stent 300 and occluder 208, can be preloaded onto the deployment wire so the surgeon does not need to assembly any components.

Embodiments of the cardiac pump system 100 provide for a simpler placement and deployment of durable LVADs by allowing percutaneous placement and deployment. This can reduce morbidity to patients at the time of the procedure, and would make subsequent heart transplantation safer and easier. Further, having shorter drive line exteriorized at the chest wall provides a safer experience for patients, as current drive line placements are prone to causing infections, thrombus, and stroke.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “r” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

What is claimed is:

1. A system for delivery and placement of an intracardiac device within a patient, comprising:
a deployment wire configured for percutaneous delivery of an intracardiac device to a cardiac chamber through a peripheral artery, the deployment wire having a proximal end portion and a distal end portion, the proximal end portion having a formed tip and the distal end portion configured to operatively connect to the intracardiac device;
a sheath configured for percutaneous insertion into a cardiac chamber wall through a chest wall, wherein the sheath includes:
a tubular body extending along an axis and having a proximal end portion, distal end portion, and a flexible portion therebetween configured for axial movement to accommodate cardiac movement during placement and delivery of the intracardiac device in the cardiac chamber,
wherein the sheath is configured to be inserted through the chest wall and into the cardiac chamber wall such that the flexible portion is within the chest wall or pericardium and proximate the chamber wall; and
wherein the distal end portion includes one or more anchoring features configured to actuate between a deployed position where the one or more anchoring features extend radially outward from the distal end portion of the sheath, and a retracted position where the one or more anchoring features are within the distal end portion; and
a snare configured to be passed through the sheath into the cardiac chamber for snaring the formed tip of the deployment wire to pull the deployment wire and intracardiac device through the peripheral artery and into the cardiac chamber.

2. The system of claim 1, wherein the proximal end portion of the sheath includes a one-way valve configured to allow insertion of deployment instruments and prevent egress of fluids from the cardiac chamber.

3. The system of claim 1, wherein the flexible portion of the sheath includes a bellows.

4. The system of claim 1, wherein with the one or more anchoring features in the retracted position, an outer circumference of the one or more anchoring features is flush with the distal end portion of the sheath.

5. The system of claim 4, wherein with the one or more anchoring features in the retracted position, the one or more anchoring features are entirely within the distal end portion of the sheath.

6. The system of claim 1, wherein the proximal end portion of the sheath includes an actuator operatively connected to the one or more anchoring features configured to actuate the one or more anchoring features to move between the retracted position and the deployed position.

7. The system of claim 6, wherein the actuator includes a push button actuator.

8. The system of claim 1, wherein the cardiac chamber is a first cardiac chamber, and further comprising:
a deployable stent configured to be operatively connected to a proximal end of the intracardiac device for securing the intracardiac device within a cardiac valve, blood vessel, or a second cardiac chamber during placement and operation of the intracardiac device within the cardiac chamber.

9. The system of claim 8, wherein the deployable stent is configured to move between a compressed position and a deployed position, wherein the deployable stent is configured to be in the compressed position during delivery of the intracardiac device, and wherein the deployable stent is configured to be deployed to the deployed position during placement and operation of the intracardiac device.

10. The system of claim 8, wherein the deployable stent comprises:
an annular portion configured to attach to and seat within a cardiac valve or the second cardiac chamber, the annular portion extending along an axis between a first end and a second end;
one or more compression members operatively connected to the annular portion and extending radially inward from the outer diameter toward the axis and positioned axially between the first end and the second end of the annular portion; and
a central bore is defined through or by the one or more compression members configured to accept the proximal end of the intracardiac device to hold the intracardiac device radially within a center of the cardiac valve or the second cardiac chamber during placement and delivery of the intracardiac device within the first cardiac chamber.

11. The system of claim 10, wherein the one or more compression members form a flexible membrane configured to allow axial and radial movement of the distal end of the intracardiac device with contraction and relaxation of the cardiac chamber.

12. The system of claim 1, further comprising an attachment member configured to be operatively connected to a distal end of the intracardiac device to secure the distal end of the intracardiac device in the cardiac chamber and proximate the chamber wall.

13. The system of claim 12, wherein the attachment member is further configured to provide a standoff distance between the distal end of the intracardiac device and the chamber wall.

14. The system of claim 1, further comprising an occluder configured to be operatively connected to a distal end of the intracardiac device to occlude an aperture in the cardiac chamber wall made by the sheath during placement and operation of the intracardiac device.

15. The system of claim 1, further comprising,
the intracardiac device, wherein the intracardiac device includes a pump having a pump inlet at a distal end and a pump outlet at a proximal end;
a deployable stent operatively connected to the proximal end of the pump for securing the pump within a cardiac valve or a second cardiac chamber during placement and operation of the pump within the cardiac chamber; and
an attachment member operatively associated with the distal end of the pump to secure the distal end of the pump in the cardiac chamber and proximate the chamber wall at a standoff distance from the chamber wall.

16. The system of claim 15, further comprising an occluder configured to occlude an aperture in the cardiac chamber wall made by the sheath during placement and operation of the intracardiac device.

17. The system of claim 16, wherein each of the pump and the occluder are operatively associated with the deployment wire such that pulling the deployment wire via the snare through the aperture in the chest wall pulls the pump and occluder through the peripheral artery and into the cardiac chamber for placement in the cardiac chamber.

18. The system of claim 17, wherein the cardiac chamber is a left ventricle, and the cardiac valve is an aortic valve, wherein the sheath is configured to be introduced through the chest wall, through a left ventricle wall, and into the left ventricle transapically, wherein the peripheral artery is the femoral artery, and wherein:
the deployment wire is configured to be introduced though and into the femoral artery and passed through to the left ventricle via the aorta and wherein the snare is configured to be introduced through the sheath transapically to snare the deployment wire whereby pulling the snare in turn externalizes the deployment wire through the sheath and pulls the pump through the femoral artery and through the ascending aorta such that the occluder is inserted into the aperture, the pump inlet is positioned in the left ventricle proximate the left ventricle wall, the pump outlet is positioned in or proximate to the ascending aorta, and the deployable stent is positioned in the ascending aorta.

19. The system of claim 18, wherein the pump further includes a drive wire configured to provide power to the pump, wherein the drive wire is externalized through the aperture with the deployment wire.

20. The system of claim 19, wherein with the pump in place, an entirety of the pump, the stent, the attachment member, and the occluder are within the heart, and wherein only the drive wire of the pump is externalized through the ventricular wall and the chest wall.
